(19) United States
(12) Patent Application Publication
(10) Pub. No.: US 2016/0109441 A1
(43) Pub. Date: Apr. 21, 2016

(54) APPARATUS FOR DETECTING BIO MATERIALS, METHOD OF FABRICATING THE APPARATUS AND METHOD OF DETECTING BIO MATERIALS USING THE APPARATUS

(71) Applicant: ELECTRONICS AND TELECOMMUNICATIONS RESEARCH INSTITUTE, Daejeon (KR)

(72) Inventors: Wan Joong KIM, Daejeon (KR); HyoYoung CHO, Daejeon (KR); CHUL HUH, Daejeon (KR); Bong Kyu KIM, Daejeon (KR); Young Jun KIM, Daejeon (KR)

(21) Appl. No.: 14/919,548

(22) Filed: Oct. 21, 2015

(30) Foreign Application Priority Data
Oct. 21, 2014 (KR) 10-2014-0142559
Sep. 30, 2015 (KR) 10-2015-0137980

(51) Int. Cl. G01N 33/543

(52) U.S. Cl. CPC G01N 33/54386

(57) ABSTRACT

Provided are an apparatus for detecting bio materials, a method of fabricating the same and a method of detecting bio materials using the same. The apparatus for detecting bio materials includes a filter, freeze dried bio material detecting materials and bio material capturing materials fixed to the inner wall of a microtube, arranged in order in the microtube. Antibodies capturing blood cells in blood are combined with the filter, each of the bio material detecting materials includes immunogolds conjugated with detecting antibodies. The apparatus for detecting bio materials is portable and perceive a small amount of the bio materials with high sensitivity.

### CROSS-REFERENCE TO RELATED APPLICATIONS

This U.S. non-provisional patent application claims priority under 35 U.S.C. §119 of Korean Patent Application Nos. 10-2014-0142559, filed on Oct. 21, 2014, and 10-2015-0137980, filed on Sep. 30, 2015, the entire contents of which are hereby incorporated by reference.

### BACKGROUND

The present disclosure herein relates to an apparatus for detecting bio materials, a method of fabricating the apparatus and a method of detecting bio materials using the apparatus, and more particularly, to a portable apparatus for detecting bio materials, a method of fabricating the apparatus and a method of detecting bio materials using the apparatus.

A biosensor is a device sensing optical or electrical signals changed according to the selective reaction and combination of a biological receptor having recognition function on a specific bio material with an analyte to be analyzed. The biosensor may check the presence of a bio material or analyze thereof qualitatively or quantitatively. As the biological receptor (that is, a sensing material), an enzyme, an antibody, DNA, etc. that may make selective reaction and combination with a specific material may be used. Bio materials are detected and analyzed by using various physicochemical methods such as electrical signal change according to the presence of an analyte, optical signal change due to the chemical reaction of a receptor and an analyte, etc. for monitoring signals.

The biosensor has been continuously developed, and a biosensor capable of diagnosing diseases using a small amount of bio materials and portable is required.

### SUMMARY

The present disclosure provides an apparatus for detecting bio materials, which is portable and may detect diseases using a small amount of bio materials.

The present disclosure also provides a method of fabricating an apparatus for detecting bio materials.

The present disclosure also provides a method of detecting bio materials using the apparatus for detecting bio materials.

The tasks for solving are not limited to the tasks mentioned above, and other tasks not mentioned above may be clearly understood from the following description by a person skilled in the art.

An embodiment of the inventive concept provides an apparatus for detecting bio materials. The apparatus for detecting bio materials includes a microtube including a first end and a second end; a filter configured to filter a body fluid provided from the first end and provided adjacent to the first end; a first reaction region disposed between the filter and the second end in the microtube and includes bio material detecting materials in a freeze dried state configured to make a specific binding with a bio material in the body fluid filtered; and a second reaction region disposed between the first reaction region and the second end and including bio material capturing materials fixed to an inner surface of the microtube configured to make a specific binding with the bio material.

In an embodiment of the inventive concept, an apparatus for detecting bio materials is provided. The apparatus for detecting bio materials includes a biosensor including a reaction part, a sensor part and a calculation part; and a stand configured to fix and support the biosensor, wherein the reaction part includes a microtube including a first end and a second end; a filter disposed adjacent to the first end and configured to filter a body fluid provided from the first end; a first reaction region disposed between the filter and the second end in the microtube and including bio material detecting materials in a freeze dried state configured to make a specific binding with a bio material in the body fluid filtered; and a second reaction region disposed between the first reaction region and the second end and including bio material capturing materials fixed to an inner surface of the microtube, configured to make a specific binding with the bio material.

In an embodiment of the inventive concept, a method of fabricating an apparatus for detecting bio materials is provided. The method of fabricating an apparatus for detecting bio materials includes preparing a hollow microtube having a first end and a second end; fixing bio material capturing materials to an inner surface of the microtube adjacent to the first end of the microtube so as to have a directional property; freeze drying the bio material detecting materials between a region where the bio material capturing materials are fixed and the second end; and disposing a filter combined with blood cell capturing antibodies between a region where the bio material detecting materials are freeze dried and the second end.

In an embodiment of the inventive concept, a method of detecting bio materials is provided. The method of detecting bio materials includes injecting a body fluid including bio materials into a first end of a microtube; passing the body fluid through a filter in the microtube for filtration; combining the bio materials in the body fluid with bio material detecting materials, respectively; combining the bio materials combined with the bio material detecting materials, respectively, with bio material capturing materials, respectively; and perceiving the presence and the amount of the bio materials fixed by the bio material capturing materials and combined with the bio material detecting materials.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a diagram for explaining an apparatus for detecting bio materials according to an embodiment of the inventive concept;

FIG. 2 is a cross-sectional view for explaining a reaction part of the apparatus for detecting bio materials in FIG. 1;

FIG. 3 is a schematic diagram for explaining the passing mechanism of bio materials through the reaction part in FIG. 2;

FIG. 4 is a schematic diagram for explaining before and after the reaction of bio material detecting materials with an enhancing solution;

FIG. 5 is a flowchart for explaining a method of fabricating an apparatus for detecting bio materials according to an embodiment of the inventive concept;

FIGS. 6A, 6B, 7A, 7B, 8A and 8B are illustrated for explaining a method of fabricating an apparatus for detecting bio materials according to an embodiment of the inventive concept; and

FIG. 9 is a flowchart for explaining a method of detecting bio materials according to an embodiment of the inventive concept.

### DETAILED DESCRIPTION

The objects, other objects, features and advantages of the inventive concept will be understood from preferred example embodiments below with reference to the accompanying drawings. The inventive concept may, however, be embodied in different forms and should not be construed as limited to the embodiments set forth herein. Rather, these embodiments are provided so that this description will be thorough and complete, and will fully convey the scope of the present inventive concept to those skilled in the art.

In the disclosure, it will be understood that when an element is referred to as being ‘on’ another element, it can be directly on the other element, or a third intervening element may be present. In the drawings, the thicknesses of elements are exaggerated for effective explanation of technical features.

Example embodiments are described herein with reference to cross-sectional views and/or plan views that are schematic illustrations of idealized example embodiments. In the drawings, the thicknesses of layers and regions may be exaggerated for effective explanation of technical contents. As such, variations from the shapes of the illustrations as a result, for example, of manufacturing techniques and/or tolerances, are to be expected. Thus, example embodiments should not be construed as limited to the particular shapes of regions illustrated herein but are to include deviations in shapes that result, for example, from manufacturing. For example, an etched region illustrated as a rectangle will, typically, have rounded or curved features. Thus, the regions illustrated in the figures are schematic in nature and their shapes are not intended to illustrate the actual shape of a region of a device and are not intended to limit the scope of the present inventive concept. It will be understood that, although the terms first, second, etc. may be used herein to describe various elements, these elements should not be limited by these terms. These terms are only used to distinguish one element from another element. Example embodiments embodied and described herein may include complementary example embodiments thereof.

The terminology used herein is for the purpose of describing particular example embodiments only and is not intended to limit the present inventive concept. As used herein, the singular forms are intended to include the plural forms as well, unless the context clearly indicates otherwise. It will be further understood that the terms “comprises” and/or “comprising,” when used in this specification, do not preclude the presence or the addition of one or more other elements.

Hereinafter, exemplary embodiments of the inventive concept will be described in detail with reference to the accompanying drawings.

In the present disclosure, bio materials are bio molecules exhibiting specific characters and may be interpreted to have the same meaning as a target molecule or an analyte. In an embodiment of the inventive concept, the bio material may be an antigen.

In the present disclosure, bio material detecting materials are bio molecules making specific bindings with the bio materials and may be interpreted to have the same meaning as a probe molecule, a receptor or an acceptor. In an embodiment of the inventive concept, the bio material detecting materials may include detecting antibodies.

In the present disclosure, bio material capturing materials are bio molecules making specific bindings with the bio materials. In an embodiment of the inventive concept, the bio material capturing material may include capturing antibodies.

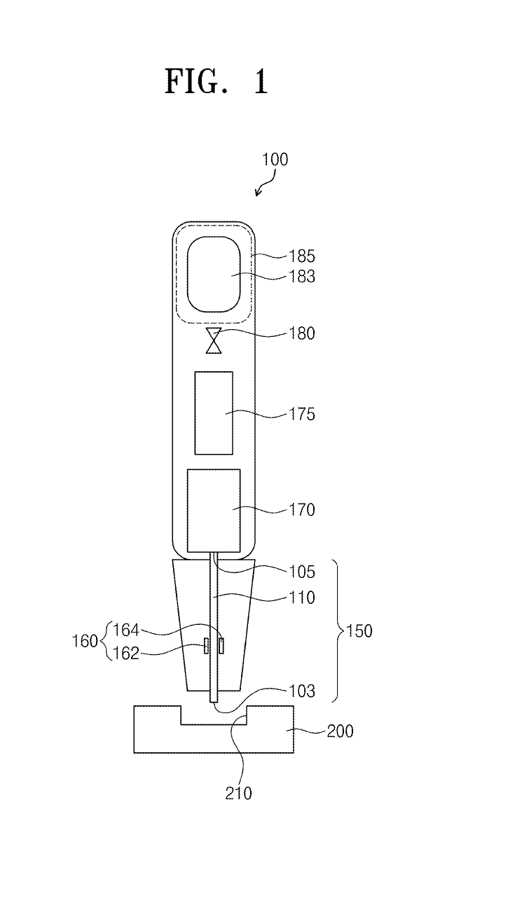

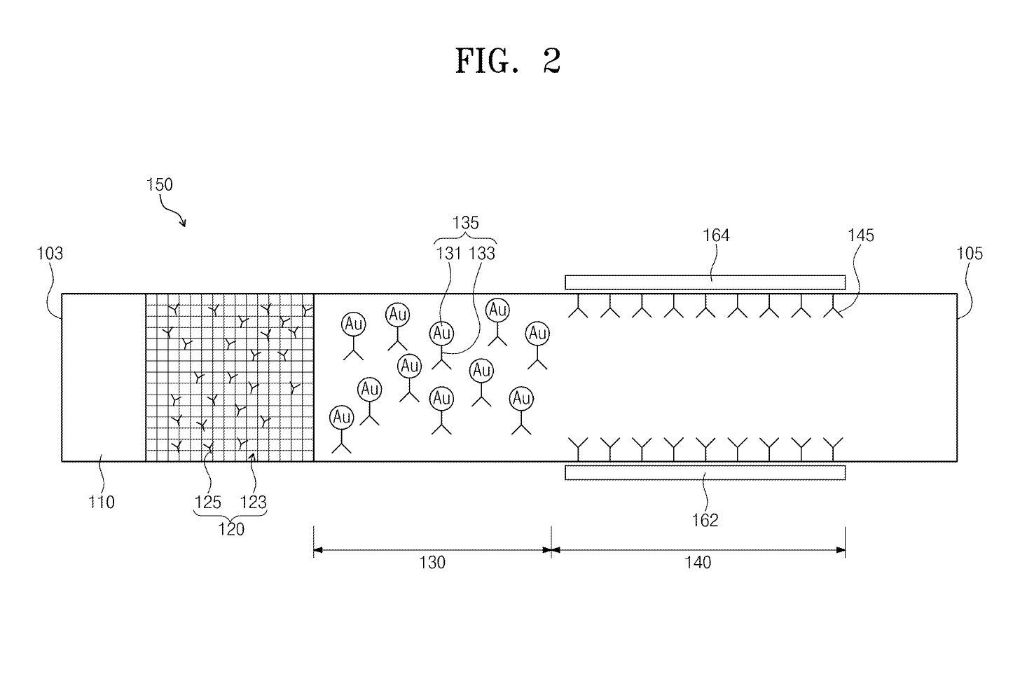

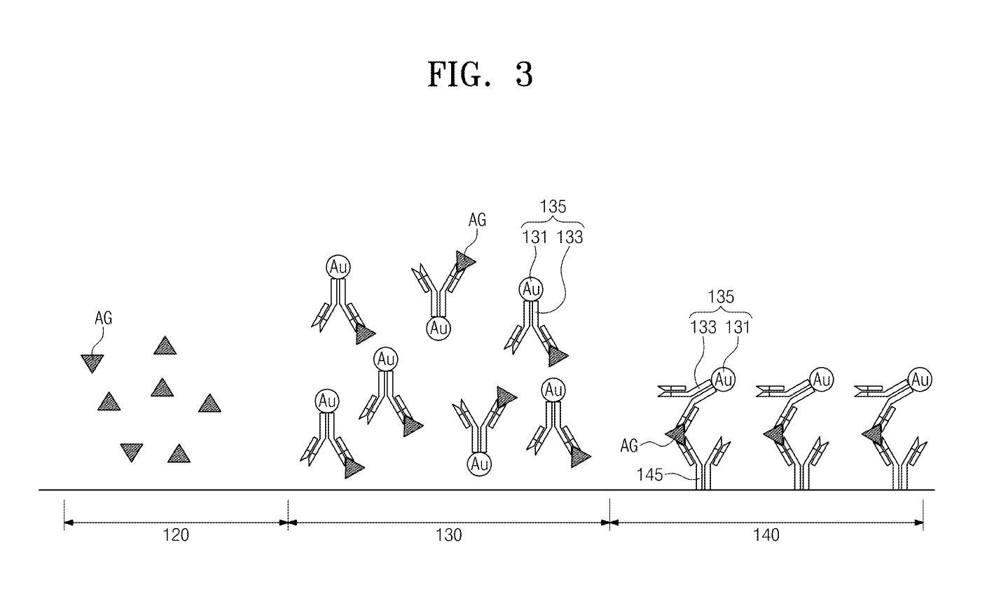

FIG. 1 is a configuration diagram for explaining an apparatus for detecting bio materials according to an embodiment of the inventive concept, FIG. 2 is a cross-sectional view for explaining a reaction part of the apparatus for detecting bio materials in FIG. 1 and FIG. 3 is a schematic diagram for explaining the passing mechanism of the bio materials through the reaction part in FIG. 2.

Referring to FIGS. 1 and 2, an apparatus for detecting bio materials may include a biosensor 100 and a stand 200. The biosensor 100 may include a reaction part 150, a sensor part 160 and a calculation part 185.

The reaction part 150 may include a hollow microtube 110 having first and second ends 103 and 105 facing to each other in an open state. According to an embodiment, the microtube 110 may have a length from about 40 to about 60 mm and a diameter from about 0.5 to about 1.5 mm. In addition, the microtube 110 may include, for example, plastics, glasses or silicone.

A solution including bio materials may be provided into the microtube 110 via the first end 103 of the microtube 110. The solution including the bio materials may be a body fluid acquired from a living matter such as blood, urine and saliva. Each of the bio materials may be one of nucleic acids, cells, viruses, proteins, organic molecules or inorganic molecules. The protein as the bio material may be one of antigens, antibodies, matrix proteins, enzymes or coenzymes. The nucleic acid as the bio material AG may include DNA, RNA, PNA, LNA or a mixture thereof. In an embodiment of the inventive concept, the solution including the bio materials may be blood. The blood includes blood cells and plasma and may include lipid metabolism products, moisture, enzymes, antigens, antibodies and protein components such as various cells. In this case, bio materials to be detected may be present mainly in the plasma.

Meanwhile, the microtube 110 may be disposed so as to be detached from the biosensor 100 and may be removed from the biosensor 100 after use. Thus, the microtube 110 may be disposable.

The reaction part 150 may further include a filter 120, a first reaction region 130 and a second reaction region 140 disposed in the microtube 110. The filter 120, the first reaction region 130 and the second reaction region 140 may be disposed from the first end 103 to the second end 105 in that order.

Referring to FIGS. 2 and 3, the filter 120 may filter blood cells from blood provided from the first end 103 of the microtube 110 and may flow only plasma including the bio materials AG through the first and second regions 130 and 140. The filter 120 may be a micro paper including pores 123 formed therethrough. The thickness of the filter 120 and the size of the pores 123 may be changed according to the size of the bio materials AG included in the blood or the amount of the blood inflowing through the filter 120. According to an embodiment of the inventive concept, blood cells may be filtered more effectively by fixing antibodies 125 for capturing blood cells to the filter 120. Methods for fixing the antibodies 125 for capturing blood cells to the filter 120 will be explained in detail hereinafter.

The first reaction region 130 may be disposed between the filter 120 and the second reaction region 140. In the first reaction region 130, freeze dried bio material detecting materials 135 may be disposed. The bio material detecting materials 135 may be randomly disposed. According to an embodiment of the inventive concept, the bio material detecting materials 135 may include immunogolds 131 conjugated with detecting antibodies 133. The detecting antibodies 133 conjugated with the immunogolds 131 may make biochemical reaction or may be combined with the bio materials AG. According to an embodiment, the amount of the immunogolds 131 conjugated with the detecting antibodies 133 may be less than or equal to about 10 ng/ml. Generally, the amount of the bio material detecting materials 135 may be considerable greater than the amount of the bio materials AG, and in this case, the bio material detecting materials 135 may attach to the bottom of the microtube 110 or may combine with an undesired material, thereby resulting in adverse effects on detection.

In the second reaction region 140, bio material capturing materials capable of making a biochemical reaction or a combination with the bio materials AG may be fixed. According to an embodiment of the inventive concept, the bio material capturing materials may be capturing antibodies 145 with a directional property that are fixed to the inner wall of the microtube 110. Generally, the antibody has a letter Y shape and is formed of the same two heavy chains (H chain) and the same two light chains (L chain). The stem of Y is formed of only the H chain, and two ends having an arm shape are formed of one H chain and one L chain. At the two ends of the arm shape, epitopes of antigens are combined. The capturing antibodies 145 have a directional property, and the stem parts of Ys may be combined with the inner wall of the microtube 110, and both arms of the capturing antibodies 145 may be provided so as to face the inside of the microtube 110. Therefore, the antigens of the bio materials AG may be combined with the capturing antibodies 145 more easily and consistently. A method of fixing the capturing antibodies 145 in the second reaction region 140 will be explained in detail hereinafter.

The sensor part 160 may be disposed adjacent to the second reaction region 140 of the reaction part 150. According to an embodiment, the sensor part 160 may be disposed at the outer wall of the microtube 110 in the second region 140. The sensor part 160 may include a light emitting device 162 providing light for the second reaction region 140 and a detecting device 164 detecting the light passed through the second reaction region 140. In an embodiment, the light emitting device 162 may include a laser diode or a light emitting diode, and the detecting device 164 may include a photo diode or a photo transistor. The sensor part 160 may measure the amount of the bio materials AG by receiving light generated from the light emitting device 162 and detecting the light at the detecting device 164, and then, perceiving the difference of the quantities of lights.

Each of the bio materials AG may make a specific binding with the bio material detecting materials 135, respectively, and the bio materials AG combined with the bio material detecting materials 135, respectively, may make specific bindings with the bio material capturing materials to be fixed in the second region 140 of the microtube 110. The light generated from the light emitting device 162 may not penetrate the immunogolds 135 of the bio material detecting materials 135, and the quantity of light received by the detecting device 164 may be less than light generated from the light emitting device 162. By using the difference of the quantities of lights, the amount of the immunogolds 131 may be measured, and since each of the immunogolds 131 are combined with the bio materials AG, respectively, the amount of the bio materials AG may be detected.

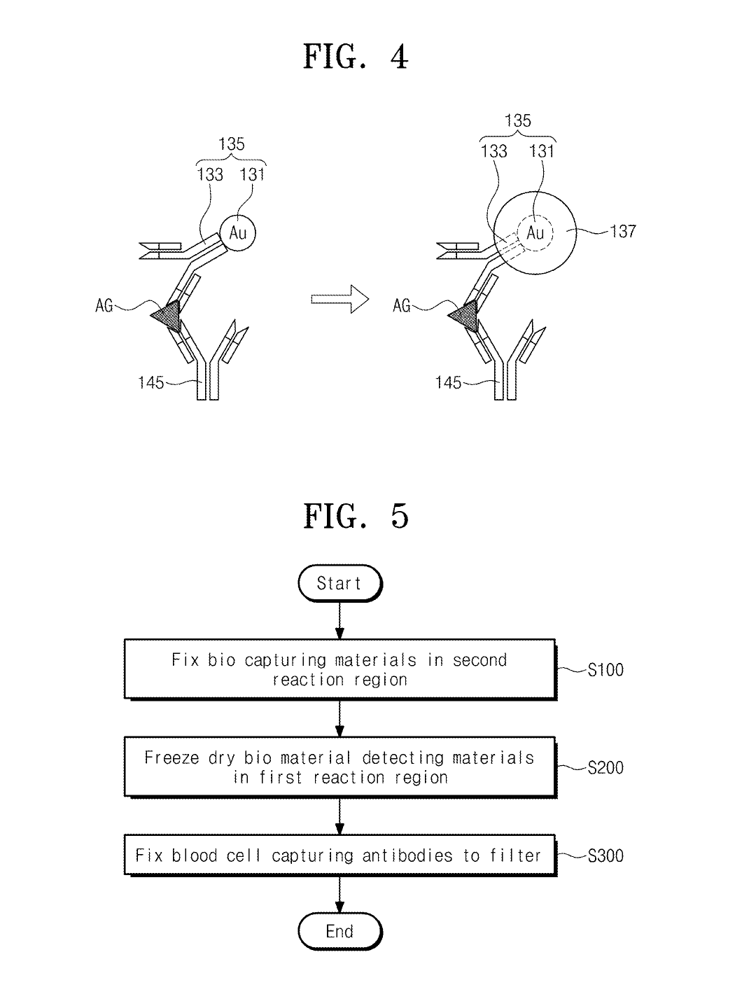

According to an embodiment of the inventive concept, the bio material detecting materials 135 combined with the bio materials AG may react with an enhancing solution to increase the sensitivity of the sensor part 160. Hereinafter this mechanism will be explained in brief FIG. 4 is a schematic diagram for explaining before and after the reaction of bio material detecting materials with an enhancing solution. Referring to FIG. 4, if the immunogolds 131 conjugated with the detecting antibodies 133 react with the enhancing solution including metallic silver, silver ions may react with the surface of the immunogolds 131 and coagulate. Thus, silver 137 may be precipitated on the surface of the immunogolds 131. The size of a metal controlling light penetration may be enlarged owing to the silver 137 precipitated at each of the immunogolds 131. Therefore, the detection of a small amount of the bio materials AG, from several to several tens pg/ml, may be possible by using the enhancing solution.

Referring to FIGS. 1 and 2, the calculation part 185 may include a circuit part (not shown) quantizing the difference of the quantities of lights perceived by the sensor part 160 and a display part 183 displaying outside the quantized value at the circuit part.

The biosensor 100 may further include a solution storing part 170 storing the enhancing solution and a cleaning solution, and a control part (not shown) opening/closing between the solution storing part 170 and the microtube 110. The solution storing part 170 may be disposed so as to communicate with the second end 105 of the microtube 110, and may provide the microtube 110 with the enhancing solution or the cleaning solution. The solution storing part 170 may include a first storing part (not shown) storing the enhancing solution and a second storing part (not shown) storing the cleaning solution. In addition, the first storing part and the microtube 110 may be shut or connected by means of a first door (not shown), and the second storing part and the microtube 110 may be shut or connected by means of a second door (not shown). The first and second doors may be controlled by the control part.

The biosensor 100 may further include a switch 180 operating the sensing process of the sensor part 160, a main board (not shown) including electronic circuits and a battery part 175.

The stand 200 may be used to fix and support the biosensor 100. For several to several tens minutes until detected results are obtained from the biosensor 100, the biosensor 100 may be accommodated in the stand 200. A recessed part 210 is formed at the upper portion of the stand 200 to provide a space for receiving an enhancing solution and a cleaning solution used in the biosensor 100.

According to an embodiment of the inventive concept, the apparatus for detecting the bio materials may include an exchangeable microtube 110, may simply diagnose diseases using a body fluid such as blood and may be portable. In addition, the diagnosis of diseases using a small amount of the bio materials AG may be possible by using an enhancing solution in the apparatus for detecting bio materials.

Hereinafter a method of fabricating the apparatus for detecting bio materials will be explained.

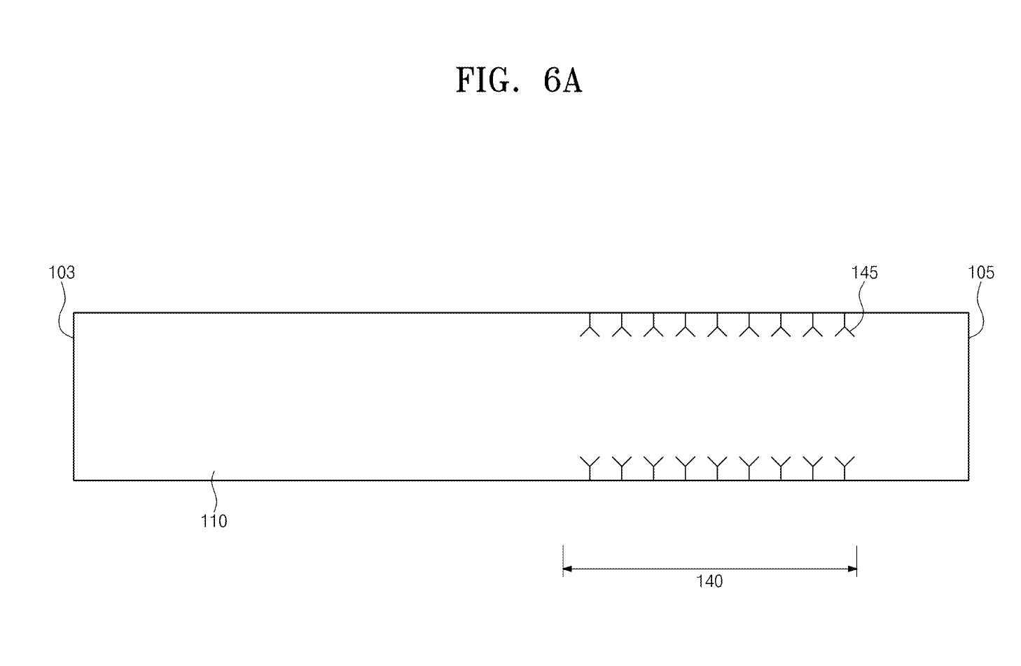

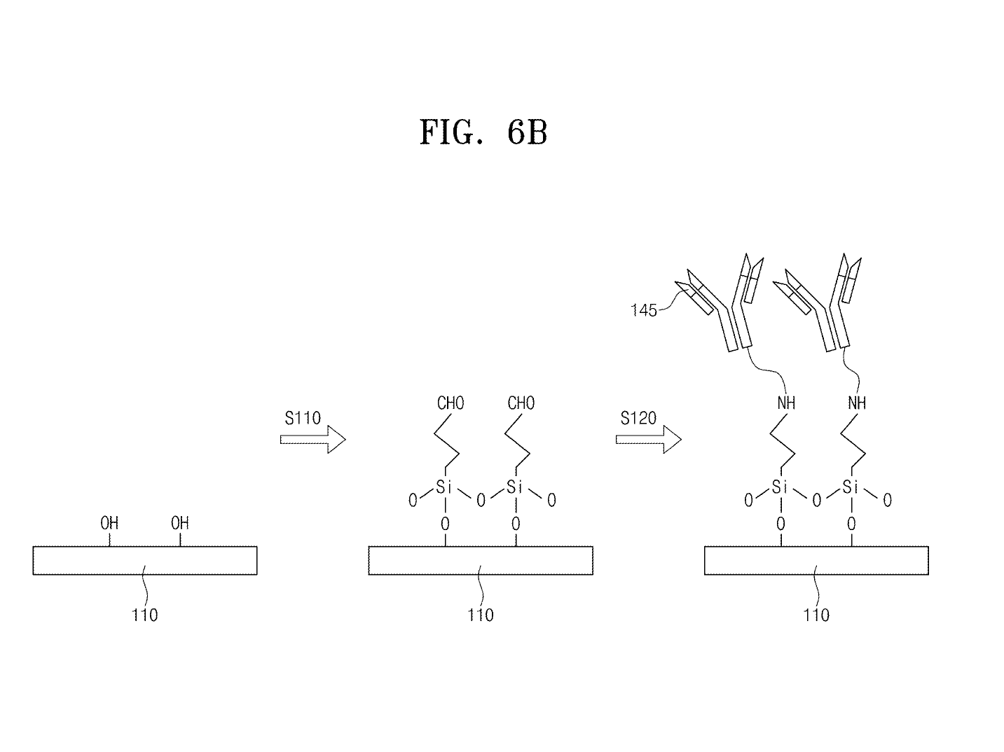

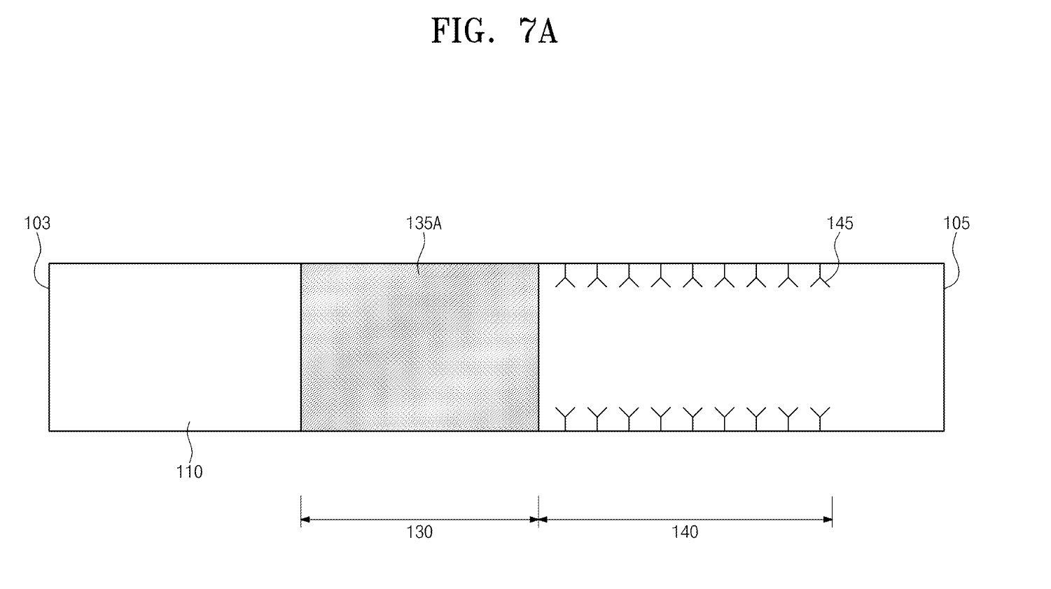

FIG. 5 is a flowchart for explaining a method of fabricating an apparatus for detecting bio materials according to an embodiment of the inventive concept, FIGS. 6A, 6B, 7A, 7B, 8A and 8B are illustrated for explaining a method of fabricating an apparatus for detecting bio materials according to an embodiment of the inventive concept. In FIGS. 5, 6A, 6B, 7A, 7B, 8A and 8B, a method of fabricating the reaction part of the biosensor in the apparatus for detecting bio materials explained in FIGS. 1 to 3 will be explained.

For convenience of explanation, the expressions of “the first” and “the second” will be explained using the same expressions of “the first” and “the second” used in FIGS. 1 to 4.

Referring to FIGS. 5, 6A and 6B, the bio material capturing materials may be fixed in the second reaction region 140 of the microtube 110.

In detail, hydroxyl groups may be introduced to the inner surface of the microtube 110 (step S110). For example, if the microtube 110 is glass, and the microtube 110 is immersed in a piranha solution (H₂SO₄:H₂O₂=7:3 (v/v)) at from about 80° C. to about 90° C. for about 30 minutes, the hydroxyl groups may be formed at the inner surface of the microtube 110.

Aldehyde groups may be combined with the hydroxyl groups combined at the inner surface of the microtube 110 (step S110). For example, an ethanol solution with 0.5 to 1.5% of aldehyde silane is injected into the microtube 110 with the hydroxyl groups formed therein. After about 1 hour, washing using ethanol was conducted. Then, baking is conducted at from about 100° C. to about 120° C. for 10 minutes for the combination of the hydroxyl groups with the aldehyde groups.

After that, capturing antibodies 145 are injected to the aldehyde groups to culture and to make combination of the capturing antibodies 145 with the inner surface of the microtube 110 (step S120).

Through the chemical combination of the capturing antibodies 145, the capturing antibodies 145 may be disposed with a directional property. As described above, each stem part of Y of the capturing antibodies 145 is combined with the aldehyde group, and both arms of each of the capturing antibodies 145 may be disposed to face the inside of the microtube 110.

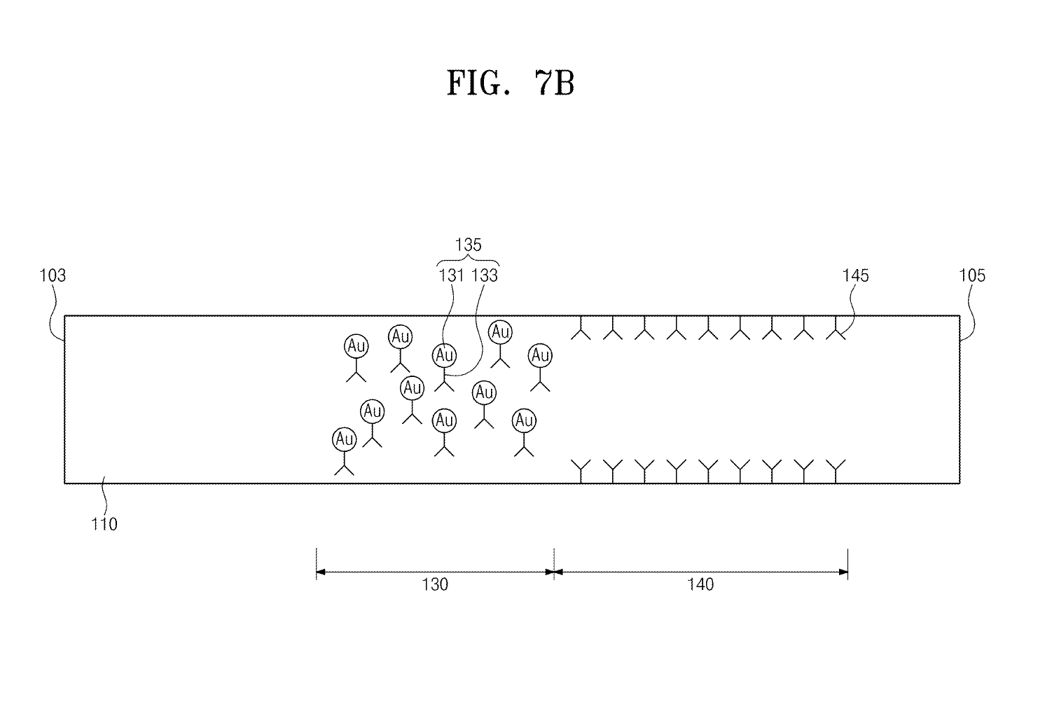

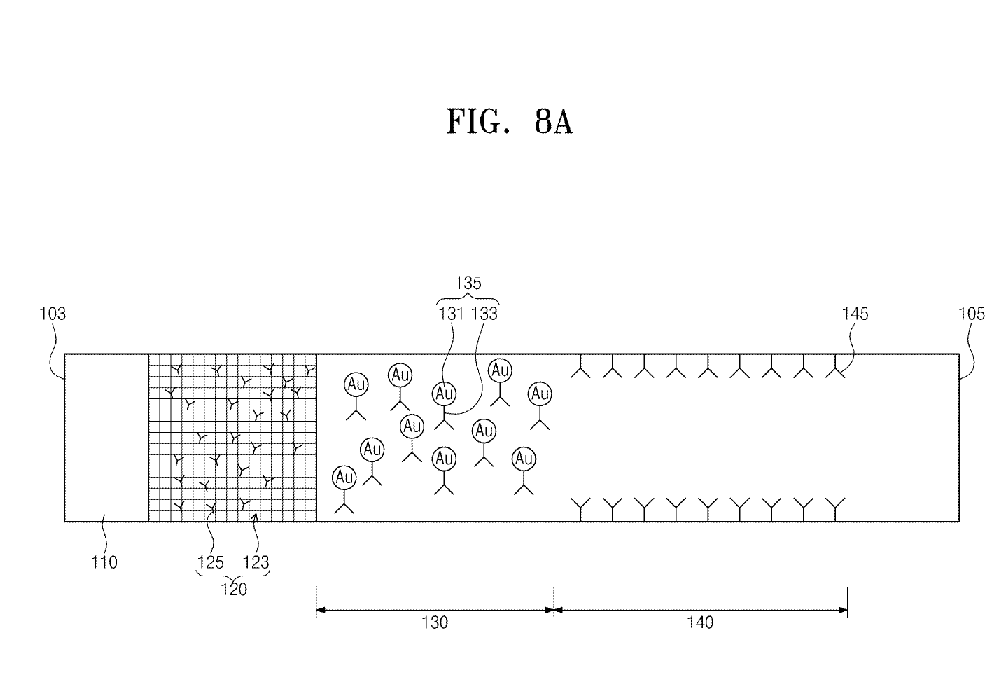

Referring to FIGS. 5, 7A and 7B, the bio material detecting materials 135 may be freeze dried in the first reaction region 130 of the microtube 110. The freeze drying may be conducted by cooling the bio material detecting materials at from about −10° C. to about −25° C. in vacuum (step S200).

More particularly, the bio material detecting materials 135 together with water including a buffer such as phosphate buffer saline (PBS) were formed into a solid state at a low temperature and disposed in the first reaction region 130 of the microtube 110. Then, the material in a solid state is freeze dried, and the PBS with water is dried to remain the bio material detecting materials 135 in the first reaction region 130. By providing the freeze dried bio material detecting materials 135 in a solid state, the modification of the detecting antibodies 133 in the bio material detecting materials 135 may be minimized.

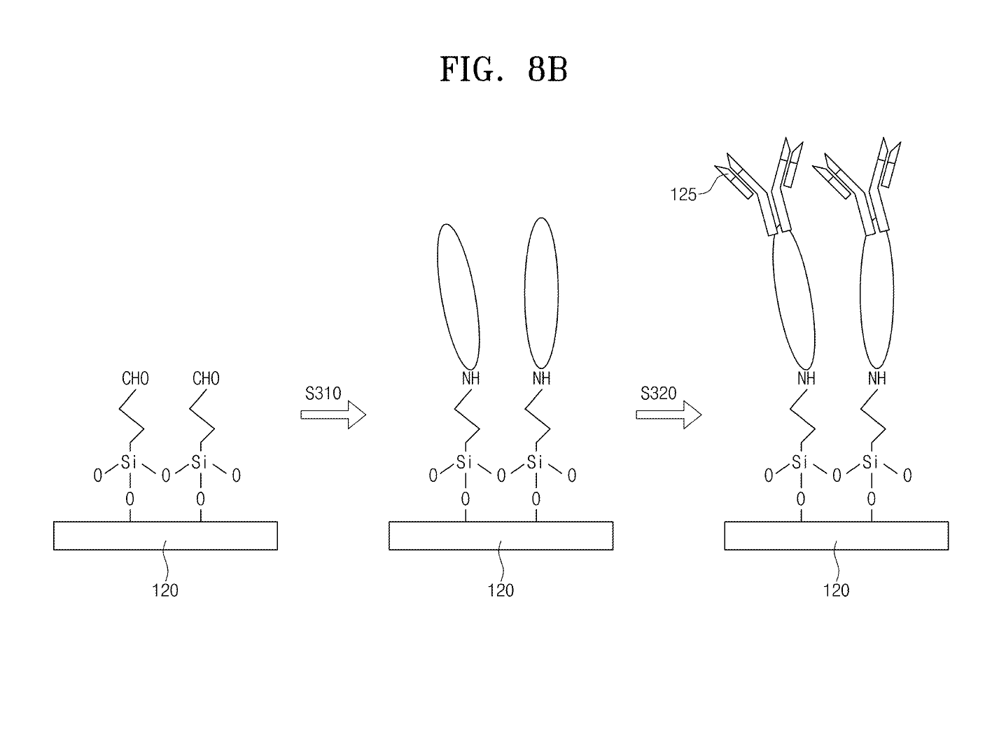

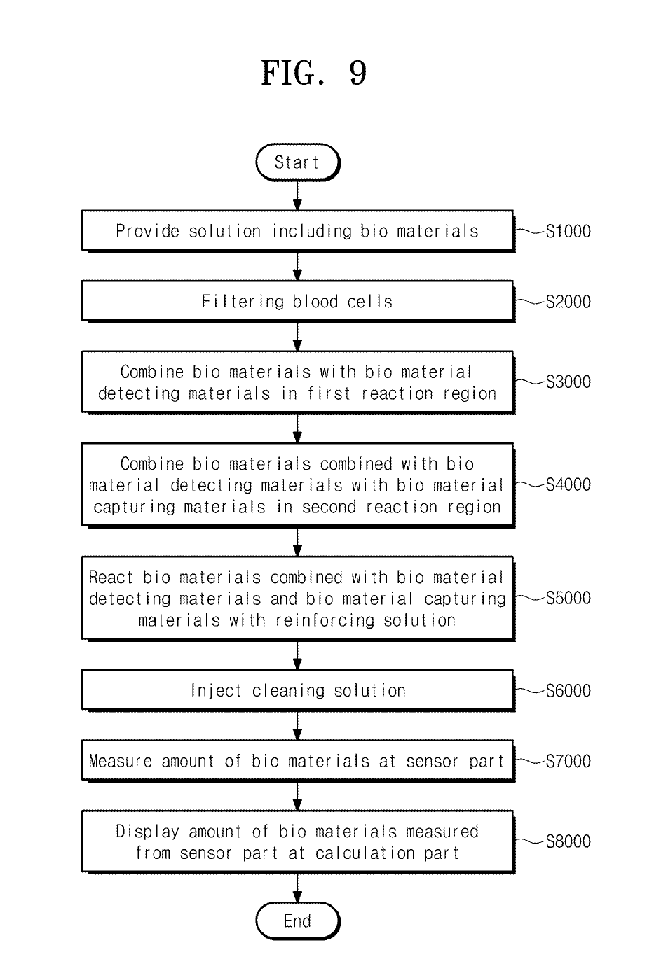

Referring to FIGS. 5, 8A and 8B, a filter 120 may be fixed adjacent to the first end 103 of the microtube 110.

The filter 120 may be a micro paper filter 120 with pores 123 formed therein. According to exemplary embodiments of the inventive concept, if the bio materials AG are included in blood, blood cells in the blood may be filtered, and plasma may be provided to the first and second reaction regions 130 and 140. Therefore, antibodies 125 capturing the blood cells may be fixed to the surface of the filter 120 (step S300). A method of fixing the antibodies 125 to the surface of the filter 120 will be explained in brief.

Referring to FIGS. 8A and 8B, aldehyde groups may be introduced to the surface of the filter 120. The aldehyde groups may be introduced to the surface of the filter by a physical method using a gas including aldehyde silane.

To the aldehyde groups combined with the surface of the filter 120, one of protein G, protein A and the complex thereof may be fixed (step S310). The antibodies 125 capturing the blood cells may be combined with one of protein G, protein A and the complex thereof (step S320).

As described above, by combining the antibodies capturing the blood cells at the surface of the filter 120, the blood cells may be primarily filtered via biological reaction or combination, and the blood cells may be secondarily filtered by the pores 123 in the filter 120, thereby effectively filtering the blood cells in the blood.

FIG. 9 is a flowchart for explaining a method of detecting bio materials according to an embodiment of the inventive concept.

Referring to FIGS. 2, 3 and 9, blood may be provided from the first end 103 of the microtube 110 of the biosensor 100 (step S1000). The blood includes bio materials AG, and the bio materials AG may be present in the plasma.

The blood cells in the blood may be filtered via the filter 120 in the microtube 110 and may move into the microtube 110. While the blood penetrates the filter 120, the blood cells may be physically filtered by the pores 123 of the filter 120, and the blood cells may be captured and filtered by the antibodies 125 at the filter 120 (step S2000).

The plasma passed through the filter 120 may move to the first reaction region 130 of the microtube 110, and the bio materials AG in the plasma may make a specific binding with the freeze dried bio material detecting antibodies 133 in the first reaction region 130 (step S3000).

Each of the bio materials AG making combination with each of the bio material detecting antibodies 133 may move to the second reaction region 140 of the microtube 110, and each of the bio materials AG may make a specific binding with each of the capture antibodies 145 fixed in the second reaction region 140 (step S4000).

The bio materials AG combined with the bio material detecting materials 135 and the capturing antibodies 145 may react with an enhancing solution (step S5000). According to an embodiment, the bio material detecting antibodies 133 may make a combination with immunogolds 131, respectively, and the enhancing solution may include metallic silver. The silver ions of the metallic silver solution may react at the surface of the immunogolds 131 and coagulate, thereby precipitating silver 147 at the surface of the immunogolds 131. Thus, the size of a metal controlling the quantity of light in each of the immunogolds 131 may be enlarged by the precipitated silver 147.

The reaction of the bio materials AG combined with the bio material detecting materials 135 and the capturing antibodies 145 and the enhancing solution may be omitted. However, the bio materials AG may be perceived with high sensitivity at the sensor part 160 using the enhancing solution.

A cleaning solution may be provided in a direction from the second end 105 to the first end 103 of the microtube 110 (step S6000). The cleaning solution may remove the bio material detecting antibodies 133 unreacted with the bio materials AG. The bio material detecting antibodies 133 unreacted with the bio materials AG may not be fixed by the fixed bio material capturing materials and may flow by the cleaning solution to the first end 103. Meanwhile, the cleaning process may be omitted as occasion demands.

The amount of the bio materials AG may be measured by the sensor part 160 (step S7000). Particularly, the bio material detecting antibodies 133 combined with the bio materials AG, respectively, are conjugated with immunogolds 131, respectively. The immunogolds 131 are a metal and do not penetrate light but shut. From the difference between light generated from the light emitting device 162 of the sensor part 160 and light received by the detecting device 164, the amount of the immunogolds 131 may be detected. The amount of the immunogolds 131 may be substantially the same as the amount of the bio materials AG, and the amount of the bio materials AG may be detected.

The amount of the bio materials AG detected by the sensor part 160 may be computed, quantized and displayed outside at the calculation part 185 (step S8000).

According to exemplary embodiments of the inventive concept, bio material detecting materials are freeze dried, and an apparatus including the same is portable. In addition, a small amount of bio materials may be perceived with high intensity via the reaction of the bio material detecting materials with an enhancing solution. Further, bio material capturing materials are aligned with a directional property on the inner surface of a microtube and may be combined with bio materials effectively.

Through the combination of blood cell capturing antibodies with a filter having pores, blood cells may be physically filtered by the pores and may be captured by the antibodies, thereby attaining effective filtering of the blood cells.

The above-disclosed subject matter is to be considered illustrative and not restrictive, and the appended claims are intended to cover all such modifications, enhancements, and other embodiments, which fall within the true spirit and scope of the inventive concept. Thus, to the maximum extent allowed by law, the scope of the inventive concept is to be determined by the broadest permissible interpretation of the following claims and their equivalents, and shall not be restricted or limited by the foregoing detailed description.

What is claimed is:

1. An apparatus for detecting bio materials, comprising:
a microtube comprising a first end and a second end;
a filter configured to filter a body fluid provided from the first end, the filter being provided adjacent to the first end;
a first reaction region disposed between the filter and the second end in the microtube, the first reaction region including bio material detecting materials in a freeze dried state, the bio material detecting materials being configured to make a specific binding with a bio material in the body fluid filtered; and
a second reaction region disposed between the first reaction region and the second end, the second reaction region including bio material capturing materials fixed to an inner surface of the microtube, the bio material capturing materials being configured to make a specific binding with the bio material.

2. The apparatus for detecting bio materials of claim 1, wherein the body fluid comprises blood, the filter comprises a plurality of pores, and antibodies configured to capture blood cells in the blood are combined with the filter.

3. The apparatus for detecting bio materials of claim 1, wherein the bio material detecting materials comprise immunogolds conjugated with detecting antibodies.

4. The apparatus for detecting bio materials of claim 1, wherein the bio material capturing materials comprise capturing antibodies, and the capturing antibodies are aligned with a directional property at the inner surface of the microtube.

5. The apparatus for detecting bio materials of claim 1, further comprising a sensor part disposed on an outer wall of the second reaction region, the sensor part being configured to perceive the presence and the amount of the bio materials using the bio material detecting materials.

6. The apparatus for detecting bio materials of claim 5, wherein the sensor part comprises:
a light emitting device configured to provide the second reaction region with light; and
a detecting device facing the light emitting device and configured to receive the light passed through the second reaction region.

7. The apparatus for detecting bio materials of claim 5, further comprising a calculation part connected with the sensor part and configured to quantify signals perceived from the sensor part and display.

8. The apparatus for detecting bio materials of claim 1, further comprising a solution storing part disposed so as to be opened and shut relative to the second end of the microtube, the solution storing part being configured to provide the second reaction region of the microtube with an enhancing solution.

9. The apparatus for detecting bio materials of claim 8, further comprising a sensor part disposed on an outer wall of the second reaction region and configured to perceive the presence and the amount of the bio materials using the bio material detecting materials,
wherein perceiving intensity of the bio materials by the sensor part increases in accordance with the provision of the enhancing solution for the second reaction region.

10. An apparatus for detecting bio materials, comprising:
a biosensor comprising a reaction part, a sensor part and a calculation part; and
a stand configured to fix and support the biosensor,
wherein the reaction part comprises:
a microtube comprising a first end and a second end;
a filter disposed adjacent to the first end and configured to filter a body fluid provided from the first end;
a first reaction region disposed between the filter and the second end in the microtube, the first reaction region comprising bio material detecting materials in a freeze dried state configured to make a specific binding with a bio material in the body fluid filtered; and
a second reaction region disposed between the first reaction region and the second end, the second reaction region comprising bio material capturing materials fixed to an inner surface of the microtube, the bio material capturing materials being configured to make a specific binding with the bio material.

11. The apparatus for detecting bio materials of claim 10, wherein the biosensor further comprises a solution storing part disposed adjacent to the reaction part,
the solution storing part comprises a cleaning solution and an enhancing solution, the cleaning solution and the enhancing solution move from the second end to the first end of the microtube, and
the stand comprises a recessed part configured to treat the cleaning solution and the enhancing solution discharged from the first end.

12. The apparatus for detecting bio materials of claim 11, wherein perceiving intensity of the bio materials by the sensor part increases in accordance with the provision of the enhancing solution for the second reaction region.

13. The apparatus for detecting bio materials of claim 10, wherein the sensor part is disposed on an outside wall of the microtube in the second reaction region, and
the sensor part further comprises a light emitting device configured to provide light for the second reaction region and a detecting device configured to receive the light passed through the second reaction region.

14. The apparatus for detecting bio materials of claim 13, wherein the calculation part is connected with the sensor part, compares quantities of lights between the light emitting device and the detecting device, quantizes and displays.

15. The apparatus for detecting bio materials of claim 10, wherein the body fluid comprises blood, the filter comprises a plurality of pores, and antibodies configured to react with the blood cells in the blood are combined with the filter.

16. The apparatus for detecting bio materials of claim 10, wherein the bio material detecting materials comprise immunogolds conjugated with detecting antibodies.

17. The apparatus for detecting bio materials of claim 10, wherein the bio material capturing materials comprise capturing antibodies, and the capturing antibodies are aligned with a directional property in an inner surface of the microtube.

18. A method of fabricating an apparatus for detecting bio materials, the method comprising:
preparing a hollow microtube having a first end and a second end;
fixing bio material capturing materials to an inner surface of the microtube adjacent to the first end of the microtube so as to have a directional property;
freeze drying the bio material detecting materials between a region where the bio material capturing materials are fixed and the second end; and
disposing a filter combined with blood cell capturing antibodies between a region where the bio material detecting materials are freeze dried and the second end.

19. The method of fabricating an apparatus for detecting bio materials of claim 18, wherein the fixing of the bio material capturing materials to the inner surface of the microtube comprises:
forming hydroxyl groups on the inner surface of the microtube;
combining aldehyde groups with the hydroxyl groups; and
combining capturing antibodies with the aldehyde groups.

20. The method of fabricating an apparatus for detecting bio materials of claim 19, wherein the forming of the hydroxyl groups on the inner surface of the microtube is conducted by immersing the microtube in a piranha solution at a temperature within a range from 80° C. to 90° C.

21. The method of fabricating an apparatus for detecting bio materials of claim 19, wherein the combining of the aldehyde groups with the hydroxyl groups comprises:
providing an ethanol solution with from 0.5 to 1.5% of aldehyde silane; and
heating to a temperature within a range from 100° C. to 125° C.

22. The method of fabricating an apparatus for detecting bio materials of claim 19, wherein the combining of the capturing antibodies with the aldehyde groups comprises inserting the capturing antibodies and culturing the capturing antibodies.

23. The method of fabricating an apparatus for detecting bio materials of claim 18, wherein the bio material detecting materials are freeze dried at a temperature within a range from −10° C. to −25° C.

24. The method of fabricating an apparatus for detecting bio materials of claim 18, wherein the combining of the blood cell capturing antibodies with the filter comprises:
introducing aldehyde groups to a surface of the filter;
fixing one of protein G, protein A and a complex thereof to the aldehyde groups; and
combining the blood cell capturing antibodies with one of the protein G, protein A and the complex thereof.

25. A method of detecting bio materials, the method comprising:
injecting a body fluid including bio materials into a first end of a microtube;
passing the body fluid through a filter in the microtube for filtration;
combining the bio materials in the body fluid with bio material detecting materials, respectively;
combining the bio materials combined with the bio material detecting materials, respectively, with bio material capturing materials, respectively; and
perceiving the presence and the amount of the bio materials, the bio materials being fixed by the bio material capturing materials and being combined with the bio material detecting materials.

26. The method of detecting bio materials of claim 25, wherein each of the bio material detecting materials comprises an immunogold conjugated with a detecting antibody.

27. The method of detecting bio materials of claim 26, further comprising:
providing an enhancing solution from a second end corresponding to the first end of the microtube,
wherein the enhancing solution comprises silver precipitated at a surface of the immunogolds conjugated with the detecting antibodies, and a size of a metal particle is enlarged by the precipitated silver.

28. The method of detecting bio materials of claim 25, further comprising:
providing a cleaning solution from the second end corresponding to the first end of the microtube to remove the bio materials uncombined with the bio material capturing materials and the bio material detecting materials uncombined with the bio materials.
